(19) United States
(12) Patent Application Publication
(10) Pub. No.: US 2026/0263186 A1
(43) Pub. Date: Sep. 10, 2026

(54) STERILE ORGANIZATION SYSTEM AND METHOD FOR RETAINING SUTURES

(71) Applicant: SterileBits, Inc., Kemah, TX (US)

(72) Inventors: Craig Ford, Kemah, TX (US); William Lawrence Patton, III, Rancho Mirage, CA (US); Guy Brent Phipps, Long Beach, CA (US); Robert James Jones, Lago Vista, TX (US)

(73) Assignee: SterileBits, Inc., Kemah, TX (US)

(21) Appl. No.: 19/459,703

(22) Filed: Jan. 26, 2026

Related U.S. Application Data
(60) Provisional application No. 63/769,406, filed on Mar. 10, 2025.

(51) Int. Cl. A61B 50/30; A61B 17/04; A61B 17/00

(52) U.S. Cl. CPC A61B 50/30; A61B 17/04; A61B 2017/00477; A61B 2050/3008; A61B 2050/314

(57) ABSTRACT

Disclosed is a sterile organization system for sutures that includes a plurality of suture sleeve pages formed from a non-clay-coated paperboard providing a smooth yet slightly textured substrate. The sterile organization system further includes various cavities, each configured with precision die-cutting or embossing and including recesses or curvatures, which are adapted to cradle individual sutures with slight retention pressure. The sterile organization system further includes a binding assembly that interconnects various suture sleeve pages in a sequential, book-style arrangement, maintained by an integrated locking mechanism. The sterile organization system also includes a sterilizable pouch to enclose the assembled pages. The cavities are arranged in a predetermined order corresponding to the sequence of suture use during surgical procedures.

### BACKGROUND

### Technical Field

The present invention generally relates to medical devices and surgical instrument organization, and more particularly to a sterile suture sleeve organizing system designed for intraoperative efficiency, facilitating the precise and rapid arrangement of sutures for complex surgical procedures.

### Description of the Related Art

Surgeries requiring high precision and stringent sterility protocols, such as cardiac and other complex procedures, often encounter challenges in managing and accessing a large number of sutures in an efficient manner. In these high-stakes settings, delays in suture retrieval or errors in suture identification may adversely affect the overall procedure, leading to potentially critical complications. Moreover, the workflow in the operating room is further complicated by the need to maintain strict sterility while ensuring that sutures are organized in an order that corresponds to the surgical steps.

Existing solutions rely on conventional methods where individual sutures are passed from a non-sterile circulating nurse to a sterile technician, or where sutures are arranged on a stand or contained within a plastic sheet featuring compartmentalized pockets. Such methodologies have been widely adopted in various surgical contexts and have historically provided a basic level of organization under sterile conditions. These solutions reflect established practices in the technical field of surgical instrument and material management.

However, these existing approaches have significant drawbacks. The manual passing of sutures is inherently time-consuming and is susceptible to issues such as mismanagement and disorganization, particularly when the number of sutures exceeds typical usage. Prior art using a plastic sheet with pockets has failed to address complications arising from variability in suture dimensions, leading to problems such as suture displacement during transport and handling, as well as reliance on a product that is no longer commercially available. These limitations compromise both efficiency and reliability in the surgical environment.

Thus, in view of the above, there is a long-felt need in the healthcare industry to address the described issues.

The approaches described in this section are approaches that could be pursued, but these are not necessarily approaches that have been previously conceived or pursued. Therefore, unless otherwise indicated, it should not be assumed that any of the approaches described in this section qualify as prior art merely by virtue of their inclusion in this section.

### SUMMARY

A sterile organization system and method for retaining one or more sutures via customized sleeve configurations are provided, as shown in and/or described in connection with at least one of the figures.

One aspect of the present disclosure relates to a sterile organization system for retaining one or more sutures via customized sleeve configurations. The sterile organization system includes a plurality of suture sleeve pages and a plurality of cavities. Each of the suture sleeve pages has a substrate formed from a non-clay-coated paperboard. The cavities are formed in each of the suture sleeve pages. Each cavity includes a geometric configuration that is defined by a precision die-cutting, such that each of the cavity is dimensioned to receive at least one of the one or more sutures. The cavities are arranged in an order corresponding to a predetermined sequence of suture use during a surgical procedure. The cavities are formed in the suture sleeve page. Each cavity has a geometric configuration defined by a precision die- cutting or embossing, such that each cavity is dimensioned to receive at least one of the one or more sutures with a slight retention pressure. The cavities are arranged in an order corresponding to a predetermined sequence of suture use during a surgical procedure.

Another aspect of the present invention relates to a method for organizing one or more sutures in a sterile state. The method includes a step of providing a plurality of suture sleeve pages fabricated from a non-clay-coated paperboard. The method further includes a step of forming a plurality of cavities in each of the plurality of suture sleeve pages. The cavities are defined by a precision die-cutting, and each is configured with recesses to cradle at least one of the one or more sutures. The method includes a step of inserting the one or more sutures into the plurality of cavities in an order corresponding to a predetermined sequence of use during a surgical procedure. The method includes a step of assembling the plurality of the suture sleeve pages into a book-style configuration using a binding assembly. The method includes a step of enclosing the plurality of suture sleeve pages within a sterilizable pouch configured to maintain sterility during one or more of an autoclaving sterilization, and a chemical sterilization.

Accordingly, one advantage of the present invention is that it provides a secure retention of sutures through optimized friction and geometrically-configured cavities, and efficient organization via a sequential binding assembly, and reliable sterility via a compatible sterilizable pouch.

Accordingly, one advantage of the present invention is that it provides an improved workflow efficiency and robust protection of essential surgical tools to deliver a novel solution that advances both the engineering and practical aspects of surgical suture management.

Other features of the example embodiments will be apparent from the drawings and from the detailed description that follows.

### BRIEF DESCRIPTION OF THE DRAWINGS

The accompanying drawings illustrate the embodiments of apparatus, systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another, and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 illustrates a block diagram of a sterile organization system for retaining one or more sutures, in accordance with at least one embodiment of the claimed subject matter;

FIG. 2 illustrates a front view of a suture sleeve page of the sterile organization system, in accordance with at least one embodiment of the claimed subject matter;

FIG. 3 illustrates a rear view of the suture sleeve page, in accordance with at least one embodiment of the claimed subject matter;

FIG. 4 illustrates a flat view of the suture sleeve page, in accordance with at least one embodiment of the claimed subject matter;

FIG. 5 illustrates a perspective view of the suture sleeve page with five cavities, in accordance with at least one embodiment of the claimed subject matter;

FIG. 6 illustrates a perspective view of the suture sleeve page with four cavities, in accordance with at least one embodiment of the claimed subject matter; and

FIG. 7 illustrates a flow diagram of a method for organizing one or more sutures in a sterile state, in accordance with embodiments of the claimed subject matter.

### DETAILED DESCRIPTION

The present disclosure is best understood with reference to the detailed figures and description set forth herein. Various embodiments of the present systems, devices, and methods have been discussed with reference to the figures. However, those skilled in the art will readily appreciate that the detailed description provided herein, including the figures are presented for explanatory purposes, and the embodiments extend beyond the currently described embodiments. For instance, the teachings and results presented in any particular described application may yield multiple alternative approaches and may be implemented in any suitable manner.

The described embodiments may be implemented manually, automatically, and/or a combination thereof. The term "method" refers to manners, means, techniques, and procedures for accomplishing any task, including, but not limited to, those manners, means, techniques, and procedures either known to the person skilled in the art or readily developed from existing manners, means, techniques, and procedures by practitioners of the art to which the embodiments pertain. Persons skilled in the art will envision many other possible variations that are within the scope of the claimed subject matter.

The present disclosure relates to a system and method for organizing sutures in a sterile, ready-to-use manner during surgical procedures. The disclosure provides a compact, secure, and efficient assembly for holding and organizing sutures using custom-designed suture sleeve pages fabricated from non-clay-coated solid bleached sulfate paperboard. The present invention leverages carefully selected material properties, tailored geometric cavity configurations, and a book-style binding assembly to ensure that sutures remain in a fixed and predetermined sequential order while also being compatible with standard sterilization processes.

Further, the present disclosure addresses the need for improved suture organization by combining practical engineering design with underlying scientific principles. The suture sleeves are engineered to not only securely hold sutures but also maintain sterility even under rigorous sterilization methods such as autoclaving or chemical sterilization. Key advantages of the present invention include a textured substrate for frictional retention, precisely formed cavities that exert a slight holding pressure, and a binding assembly that supports an organized “book” configuration, all of which work in concert to enhance the efficiency and reliability of suture handling in sterile environments.

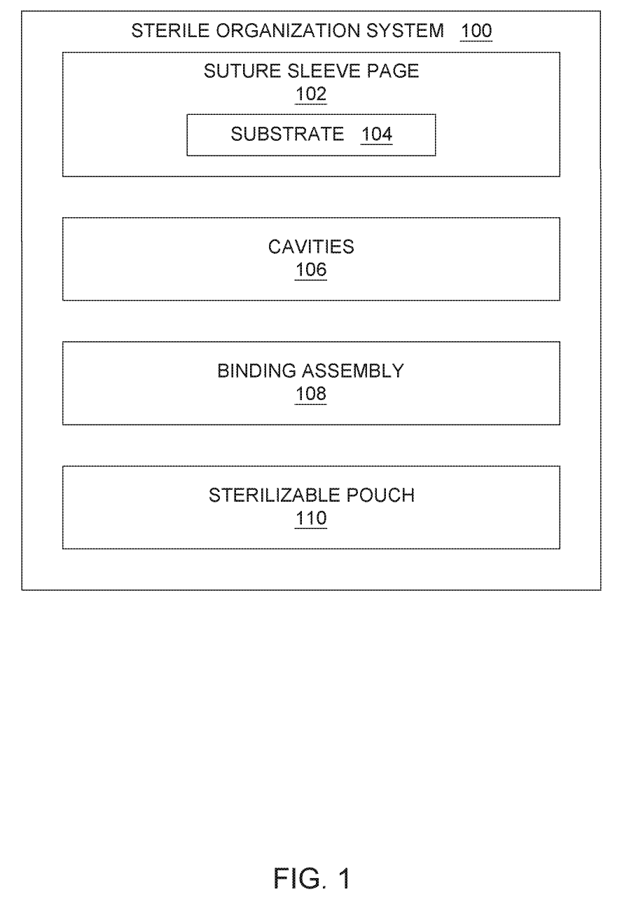

FIG. 1 illustrates a block diagram of a sterile organization system 100 for retaining one or more sutures, in accordance with at least one embodiment of the claimed subject matter. The sterile organization system includes a plurality of suture sleeve pages 102, a plurality of cavities 106, a binding assembly 108, and a sterilizable pouch 110. Each of the suture sleeve pages 102 has a substrate 104 formed from a non-clay-coated paperboard. In an embodiment, the non-clay-coated paperboard is a solid bleached sulfate paperboard.

The cavities 106 are formed in each of the suture sleeve pages 102. Each cavity 106 includes a geometric configuration that is defined by a precision die-cutting, such that each of the cavity 106 is dimensioned to receive at least one of the one or more sutures. The cavities 106 are arranged in an order corresponding to a predetermined sequence of suture use during a surgical procedure. The cavities 106 are formed in the suture sleeve page 102. Each cavity 106 has a geometric configuration defined by a precision die-cutting or embossing, such that each cavity 106 is dimensioned to receive at least one of the one or more sutures with a slight retention pressure. The cavities 106 are arranged in an order corresponding to a predetermined sequence of suture use during a surgical procedure. In an embodiment, each of the cavities 106 is provided with at least one recess or curvature formed therein for cradling at least one of the one or more sutures. In an embodiment, the substrate 104 further includes a plurality of custom-configured regions defined by a distinct surface texturing at an interface between the one or more sutures and the substrate 104 when at least one of the one or more sutures is positioned within a corresponding cavity.

The binding assembly 108 is configured to interconnect the plurality of the suture sleeve pages that are bound together to form a book-like configuration. In an embodiment, the binding assembly 108 is configured to permit individual page flipping while maintaining a sequential arrangement. In an embodiment, the binding assembly may include at least one ring member configured to secure the suture sleeve pages in a flexible yet orderly book-style configuration.

According to an additional embodiment, the binding assembly 108 incorporates a locking mechanism to retain the sequential arrangement of the suture sleeve pages during handling and storage. Additional embodiments may also modify the geometry or surface texturing of the cavities to accommodate variations in suture sizes, shapes, and materials. In an alternative embodiment, binding methods while still maintaining organized access, and the sterilizable pouch may be formed from a range of polymeric materials that exhibit resistance to degradation under sterilization conditions. Such variations remain within the scope of the invention as they continue to benefit from the fundamental technical features and principles disclosed herein.

The sterilizable pouch 110 is adapted to receive and enclose the plurality of suture sleeve pages when assembled in the book-like configuration. In an embodiment, the sterilizable pouch 110 is fabricated from a material capable of withstanding autoclaving or chemical sterilization processes. In an embodiment, the sterilizable pouch 110 is formed from a polymeric material resistant to degradation under autoclaving conditions.

According to an additional embodiment, the sterilizable pouch 110 is fabricated from a polymeric material known to withstand harsh sterilization processes such as autoclaving or chemical sterilization while maintaining an effective sterile barrier. The sterilizable pouch 110 may function as an outer containment unit, preserving both the order of the sutures and the sterility of the assembly until the moment of use. The integration of the binding assembly 108 within the pouch ensures that the overall configuration is maintained throughout storage and handling.

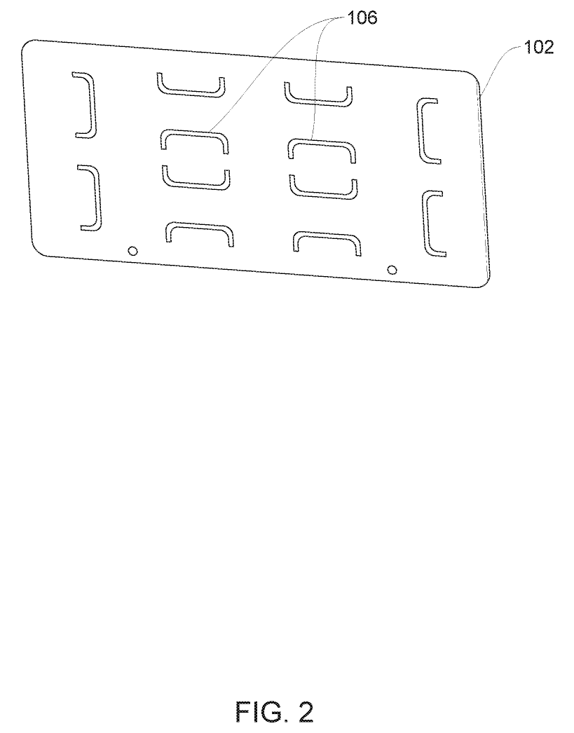

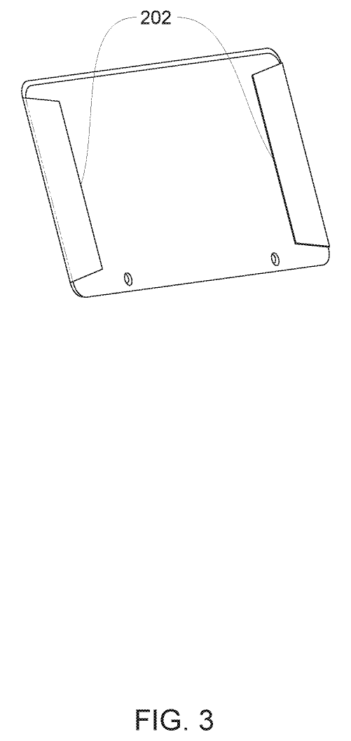

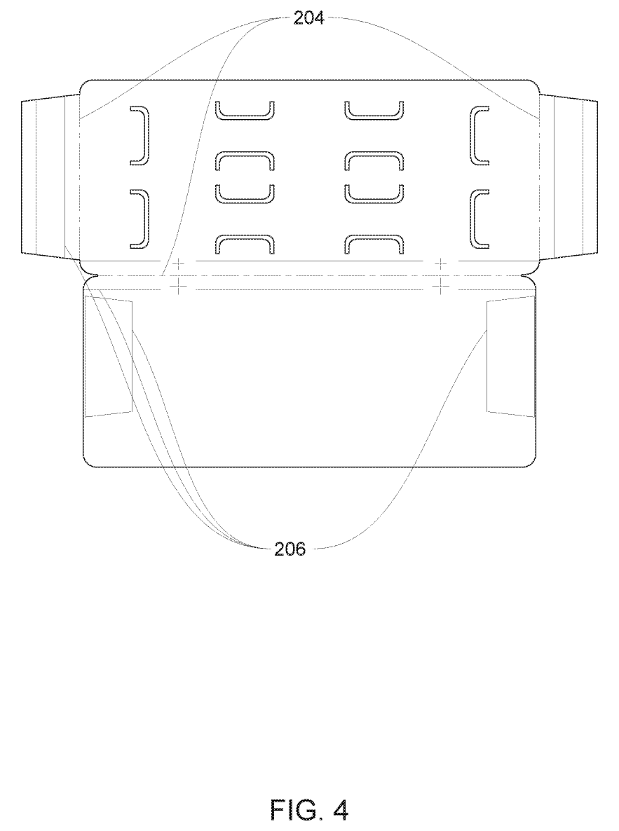

Further, FIG. 2 is explained in conjunction with FIGS. 2-4. FIG. 2 illustrates a front view of the suture sleeve page 102 of a sterile organization system 100, showing die-cut cavities or slot openings 106 formed in the substrate 104 of non-clay coated paperboard. The cavities 106 are arranged in a predetermined sequence and are configured to receive and retain one or more sutures during a surgical procedure. FIG. 3 illustrates a rear view of the suture sleeve page 102 of FIG. 2 in a folded or in-use configuration, showing opposing glued flap portions 202 that define an envelope-like structure. FIG. 3 further illustrates binding apertures or binding interface regions configured to cooperate with the binding assembly 108 for interconnecting multiple suture sleeve pages into a book-like configuration. FIG. 4 illustrates a flat, and unfolded view of the suture sleeve page 102 before use or assembly. As shown, the suture sleeve page 102 includes a substrate 104 that is rectangular. The cavities or slot openings 106 are formed through the substrate 104 by precision die-cutting to define multiple cavities arranged in a predetermined layout. The cavities 106 are configured to receive and retain sutures of varying sizes and thicknesses, while the surrounding substrate material provides structural rigidity suitable for handling and sterilization. Fold lines 204 and glue regions 206, where present, indicate areas used to form the sleeve into an in-use configuration when assembled.

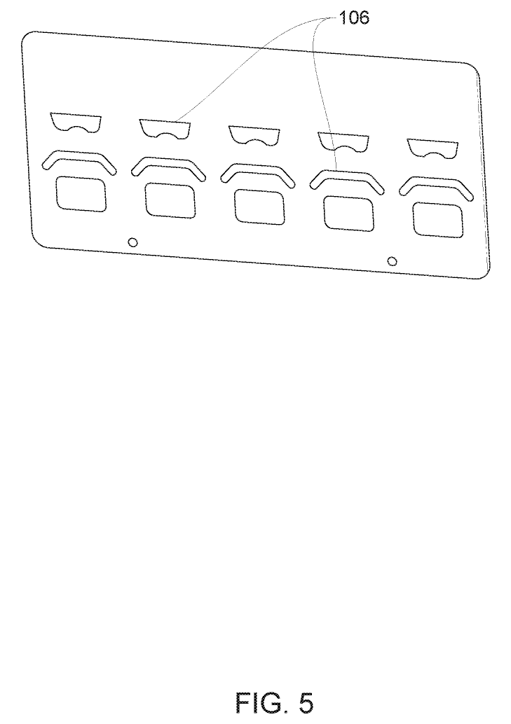

FIG. 5 illustrates a perspective view of a suture sleeve page 102 having five cavities 106 formed therein, in accordance with one embodiment. FIG. 5 is explained in conjunction with FIG. 1-4. In this configuration, the plurality of cavities or slot openings 106 collectively define five distinct cavities, each shaped to receive a corresponding suture. The geometric configuration of the cavities, including curved and recessed slot portions, cooperates with the inherent surface texture of the paperboard substrate to cradle the sutures and resist unintended movement during transport and handling. The spacing and arrangement of the cavities are selected to facilitate organized placement of sutures in a sequence corresponding to a surgical workflow.

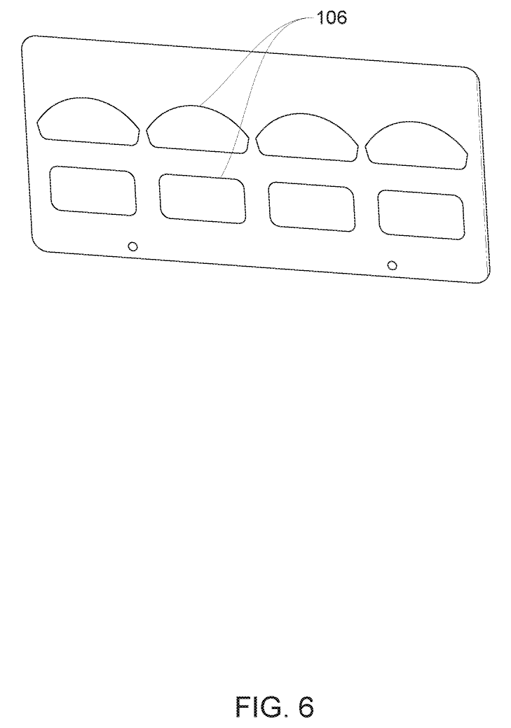

FIG. 6 illustrates a perspective view of an alternative embodiment of the suture sleeve page 100 having four cavities. FIG. 6 is explained in conjunction with FIG. 1-5. Like the embodiment of FIG. 5, the cavities are defined by slot openings 104 formed through the substrate 102. The reduced number of cavities demonstrates that the suture sleeve page 100 may be configured with different cavity counts while maintaining the same functional principles of suture retention, organization, and sterility. The embodiment of FIG. 6 is particularly suited for procedures requiring fewer sutures or larger suture formats, while remaining compatible with binding into a book-like configuration and placement within a sterilizable pouch.

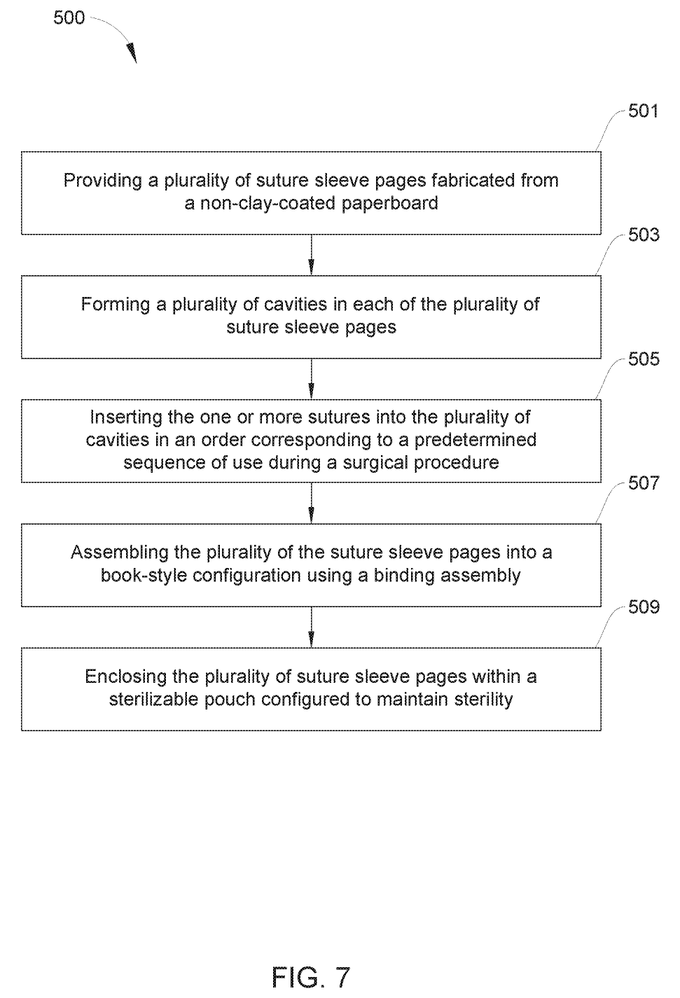

FIG. 7 illustrates a flow diagram of a method 500 for organizing one or more sutures in a sterile state, in accordance with embodiments of the claimed subject matter. FIG. 7 is explained in conjunction with FIG. 1-6. The method 500 includes a step 501 of providing a plurality of suture sleeve pages fabricated from a non-clay-coated paperboard. The method 500 further includes a step 503 of forming a plurality of cavities in each of the plurality of suture sleeve pages. The cavities are defined by a precision die-cutting, and each is configured with recesses to cradle at least one of the one or more sutures. In an operational embodiment, the step of forming the plurality of custom-designed cavities may include computer-aided designing of a cavity layout and subsequently die-cutting the substrate to define the cavities. The method 500 includes a step 505 of inserting the one or more sutures into the plurality of cavities in an order corresponding to a predetermined sequence of use during a surgical procedure. The method 500 includes a step 507 of assembling the plurality of the suture sleeve pages into a book-style configuration using a binding assembly. The method 500 includes a step 509 of enclosing the plurality of suture sleeve pages within a sterilizable pouch configured to maintain sterility during one or more of an autoclaving sterilization, and a chemical sterilization. In an embodiment, the method may include a step of integrating a locking mechanism with the binding assembly to retain the sequential arrangement of the suture sleeve pages during handling and storage.

In operation, the suture sleeve pages are provided as custom-designed pages configured to retain sutures of varying sizes and thicknesses in an organized and sterile manner. Each suture sleeve is formed from a non-clay-coated solid bleached sulfate paperboard substrate, which provides sufficient rigidity and surface characteristics suitable for surgical handling and sterilization. The plurality of slots formed in the substrate defines cavities through which sutures may be inserted. The geometric configuration of the cavities, in combination with the inherent surface texture of the paperboard, is configured to resist unintended movement of the sutures during transport, storage, and handling before use. After loading, multiple suture sleeves may be bound together in a book-like configuration, placed within a sterilizable pouch, and subjected to a sterilization process to maintain the sutures in a sterile condition until surgical use.

Thus, the present invention provides enhanced stability during transportation and handling by securing sutures within custom-shaped, textured cavities featured on the suture sleeve pages. The integration of the book-style configuration and the sterilizable pouch facilitates rapid retrieval during surgical procedures while maintaining a high level of sterility. The technical features, including precise dimensional control of the cavities, optimized frictional enhancement through surface texturing, and an organized, sequential arrangement, collectively contribute to improved workflow, reduced procedural errors, and enhanced reliability of suture management within a sterile field.

Unless otherwise defined, all terms (including technical and scientific terms) used in this disclosure have the same meaning as commonly understood by one of ordinary skill in the art to which this disclosure belongs. It is to be understood that the phrases or terms used with the present inventive subject matter are for description and not limitation. As will be appreciated by one of the skills in the art, the present disclosure may be embodied as a device, system, or method. The present systems, devices, and methods have been described above with reference to specific examples, however, other embodiments and examples than the above description are equally possible within the scope of the claimed subject matter. The scope of the disclosure may only be limited by the appended patent claims. Even though modifications and changes may be suggested by the persons skilled in the art, the inventors and applicants intend to embody within the patent warranted hereon all the changes and modifications as reasonably and properly come within the scope of the contribution of the inventors and applicants to the art. The scope of the embodiments of the inventive subject matter is ascertained with the claims as submitted at the time of filing the complete specification.

What is claimed is:

1. A sterile organization system for retaining one or more sutures, comprising:
a plurality of suture sleeve pages having a substrate formed from a non-clay-coated paperboard; and
a plurality of cavities formed in each of the suture sleeve pages, wherein each cavity comprising a geometric configuration defined by a precision die-cutting, such that each of the cavities are dimensioned to receive at least one of the one or more sutures, wherein the plurality of cavities are arranged in an order corresponding to a predetermined sequence of suture use during a surgical procedure.

2. The sterile organization system of claim 1, further comprising:
a binding assembly configured to interconnect the plurality of the suture sleeve pages that are bound together to form a book-like configuration; and
a sterilizable pouch adapted to receive and enclose the plurality of suture sleeve pages when assembled in the book-like configuration.

3. The sterile organization system of claim 2, wherein the binding assembly is configured to permit individual page flipping while maintaining a sequential arrangement.

4. The sterile organization system of claim 2, wherein the sterilizable pouch is fabricated from a material capable of withstanding autoclaving or chemical sterilization processes.

5. The sterile organization system of claim 2, wherein the binding assembly comprises at least one ring member configured to secure the suture sleeve pages in a flexible yet orderly book-style configuration.

6. The sterile organization system of claim 2, wherein the sterilizable pouch is formed from a polymeric material resistant to degradation under autoclaving conditions.

7. The sterile organization system of claim 1, wherein each of the cavities is provided with at least one recess or curvature formed therein for cradling at least one of the one or more sutures.

8. The sterile organization system of claim 1, wherein the substrate further comprises a plurality of custom-configured regions defined by a distinct surface texturing at an interface between the one or more sutures and the substrate when at least one of the one or more sutures is positioned within a corresponding cavity.

9. The sterile organization system of claim 1, wherein the non-clay coated paperboard is a solid bleached sulfate paperboard.

10. A method for organizing one or more sutures in a sterile state, comprising:
providing a plurality of suture sleeve pages fabricated from a non-clay-coated paperboard;
forming a plurality of cavities in each of the plurality of suture sleeve pages, wherein the cavities are defined by a precision die-cutting, and each is configured with recesses to cradle at least one of the one or more sutures;
inserting the one or more sutures into the plurality of cavities in an order corresponding to a predetermined sequence of use during a surgical procedure;
assembling the plurality of the suture sleeve pages into a book-style configuration using a binding assembly; and
enclosing the plurality of suture sleeve pages within a sterilizable pouch configured to maintain sterility during one or more of an autoclaving sterilization, and a chemical sterilization.
